(19) United States
(12) Patent Application Publication
(10) Pub. No.: US 2003/0143186 A1
(43) Pub. Date: Jul. 31, 2003

(54) Protease enzyme inhibitors

(75) Inventors: Todd Laurence Underiner, Cincinnati, OH (US); Renae Dianna Fossum, Middletown, OH (US); John McMillan McIver, Cincinnati, OH (US)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company, Cincinnati, OH (US)

(21) Appl. No.: 10/285,071

(22) Filed: Oct. 31, 2002

Related U.S. Application Data
(60) Provisional application No. 60/338,038, filed on Nov. 13, 2001.

(51) Int. Cl. A61K 31/74; C08G 59/14

(52) U.S. Cl. 424/78.18; 525/523

(57) ABSTRACT

The present invention relates to compounds having the general formula:

[Poly][(L)_{z}—[R—CHO]┐_{y}

wherein [Poly] is a water-soluble, non-peptidic polymer component, L is an optionally present linking group, and RCHO is a substrate capable of interacting with one or more protease enzymes to reversibly inhibit said enzyme, and wherein said substrate comprises a peptide or peptide-like aldehyde. The index z is 0 when linking groups are absent and z is 1 when linking groups are present. The index y is at least 1. The inhibitors of the present invention are suitable for use in personal care compositions, inter alia, body washes, shampoos, and skin creams.

### CROSS REFERENCE TO RELATED APPLICATIONS

This application claims priority under Title 35, United States Code 119(e) from Provisional Application Serial No. 60/338,038 filed Nov. 13, 2001.

### FIELD OF THE INVENTION

The present invention relates to an enzyme inhibitor conjugate which is capable of reversibly inhibiting one or more enzymes. The inhibitors of the present invention are used in formulations comprising one or more enzymes wherein the activity of said enzyme is suppressed by said inhibitor until said formulation is utilized by the consumer.

### BACKGROUND OF THE INVENTION

Enzymes make up the largest class of naturally occurring proteins and have found wide utility in consumer products. The specificity of enzymatic activity and their almost inexhaustible catalytic behavior, for example, their ability to hydrolyze proteins, has been exploited by incorporating both naturally occurring and genetically engineered enzymes into cleaning compositions. Of the various types of enzymes, proteases are ubiquitous and have a wide range of applications.

Until now, proteases have been utilized to a lesser extent in personal care compositions wherein each ingredient must bear the burden of efficacy and utility. Enzymes, especially protease enzymes, lead to inherent composition instability in that the enzyme can and will attack any ingredient which can serve as a substrate. Controlling the ability of an enzyme to attack other composition ingredients while not inhibiting the ability of the enzyme to function once applied, has been a problem confronting formulators of enzyme-containing, especially protease enzyme-containing, personal care compositions.

There is therefore, a long felt need in the art for a means for controlling the activity of protease enzymes while the enzymes are contained in a product matrix. The means for controlling the enzyme must be efficient, stable, and highly reversible once the product matrix is applied. The present invention satisfies a long felt need in that it provides a protease enzyme inhibitor which serves as a means for holding an enzyme's catalytic activity in abeyance until a time wherein the formulator or consumer desires the enzyme to begin functioning.

### SUMMARY OF THE INVENTION

The present invention meets the aforementioned needs in that it has been surprisingly discovered that enzyme activity can be reversibly inhibited by certain polymer conjugates, said conjugates suitable for use in compositions which contact human skin, as well as inert surfaces. It has been surprisingly discovered that the high molecular weight of the novel conjugates described herein prevents the enzyme-active substrate portion from penetrating into the skin, but does not inhibit or abate the ability of the substrate component from interacting with the targeted enzyme. In addition, the selection of various substituent groups which comprise the substrate portion allows the formulator to modulate the level of inhibition and prepare compounds which inhibit a specific species of enzyme or, in another iteration of the present invention, prepare conjugates which inhibit a wide range of enzymes.

The present invention relates to new compositions of matter which are capable of inhibiting enzymatic activity and which are compatible with exposure to human tissue. The present invention relates to compounds having the general formula:

[Poly][(L)_{z}—[R—CHO]┐_{y}

wherein [Poly] is a water-soluble, non-peptidic polymer component, L is an optionally present linking group, and RCHO is a substrate capable of interacting with one or more enzymes to reversibly inhibit said enzyme, and wherein said substrate comprises a peptide or peptide-like aldehyde. The index z is 0 when linking groups are absent and z is 1 when linking groups are present.

These and other objects, features, and advantages will become apparent to those of ordinary skill in the art from a reading of the following detailed description and the appended claims. All percentages, ratios and proportions herein are by weight, unless otherwise specified. All temperatures are in degrees Celsius (° C.) unless otherwise specified. All documents cited are in relevant part, incorporated herein by reference.

### DETAILED DESCRIPTION OF THE INVENTION

As described herein, each of the general aspects of the present invention will themselves have a plurality of aspects relating to variations of the main compound structure and which will be set forth and further described herein by the various embodiments encompassed within these various aspects. The non-limiting embodiments described herein of each aspect will be further characterized and illustrated by various non-limiting iterations relating to each embodiment. Specific examples of the herein described iterations will provide suitable enablement to make and to use all aspects of the present invention.

The term “substituted” is used throughout the specification and for the purposes of the present invention the term “substituted” is defined as “replacement of a hydrogen atom, two hydrogen atoms, or three hydrogen atoms from a carbon atom to form a moiety, or the replacement of hydrogen atoms from adjacent carbon atoms to form a moiety.” For example, a substituted unit that requires a single hydrogen atom replacement includes halogen, hydroxyl, and the like. A two hydrogen atom replacement includes carbonyl, oximino, and the like. Three hydrogen replacement includes cyano, and the like. The term substituted is used throughout the present specification to indicate that a moiety, inter alia, aromatic ring, alkyl chain, can have one or more of the hydrogen atoms replaced by a substituent. For example, 4-hydroxyphenyl is a “substituted aromatic carbocyclic ring”, and 3-guanidinopropyl is a “substituted C₃ alkyl unit.”

The following are non-limiting examples of moieties, which can replace hydrogen atoms on carbon to form a “substituted hydrocarbyl” unit:

i) —NHCOR³⁰;

ii) —COR³⁰;

iii) —COOR³⁰;

iv) —COCH═CH₂;

v) —C(═NH)NH₂;

vi) —N(R³⁰)₂;

vii) —NHC₆H₅;

viii) ═CHC₆H₅;

ix) —CON(R³⁰)₂;

x) —CONHNH₂;

xi) —NHCN;

xii) —OCN;

xiii) —CN;

xiv) F, Cl, Br, I, and mixtures thereof;

xv) ═O;

xvi) —OR³⁰;

xvii) —NHCHO;

xviii) —OH;

xix) —NHN(R³⁰)₂;

xx) ═NR³⁰;

xxi) ═NOR³⁰;

xxii) —NHOR³⁰;

xxiii) —CNO;

xxiv) —NCS;

xxv) ═C(R³⁰)₂;

xxvi) —SO₃M;

xxvii) —OSO₃M;

xxviii) —SCN;

xxix) —P(O)H₂;

xxx) —PO₂;

xxxi) —P(O)(OH)₂;

xxxii) —SO₂NH₂;

xxxiii) —SO₂R³⁰;

xxxiv) —NO₂;

xxxv) trihalomethyl having the formula: —CF₃, —CCl₃, —CBr₃;

xxxvi) and mixtures thereof;

wherein R³⁰ is hydrogen, C₁-C₂₀ linear or branched alkyl, C₆-C₂₀ aryl, C₇-C₂₀ alkylenearyl, and mixtures thereof; M is hydrogen, or a salt forming cation.

The present invention is directed to a polymer conjugate comprising a substrate which is a peptide aldehyde or peptide-like aldehyde residue. The compounds of the present invention have the general formula:

[Poly][(L)_{z}—[R—CHO]┐_{y}

wherein [Poly] is a water-soluble, non-peptidic polymer component, L is an optionally present linking group, and RCHO is a substrate capable of interacting with one or more enzymes to reversibly inhibit said enzyme, and wherein said substrate comprises a peptide or peptide-like aldehyde. As can be seen from the formula above, the [Poly] unit is bonded to one or more enzyme substrate units at the opposite terminus from the aldehyde functional group. When a linking unit is present, the [Poly] unit will be attached to the linking unit, L, which will be attached similarly to the enzyme substrate unit. The index y indicates the number of aldehyde comprising units which are bonded to the polymer component. For embodiments of the present invention wherein the index y>1, not all —RCHO units must be attached by the same linking unit if a linking unit is present, or alternatively, some —RCHO units may be attached via an L unit while others are directly bonded to the [Poly] component.

The first aspect of the overall invention relates to conjugates comprising one substrate unit (y=1), said compounds having the formula:

[Poly][(L)_{z}—[R—CHO]

of which there are two embodiments, one comprising a linking unit and one having no linking unit.

The second aspect of the overall invention relates to conjugates comprising two substrate units (y=2), said compounds having the formula:

[Poly][(L)_{z}—[R—CHO]┐₂

one embodiment of which includes conjugates wherein the aldehyde comprising unit is connected at the terminal ends of a [Poly] backbone, said conjugates having the formula:

[OHC—R]—(L)_{z}—[Poly]—(L)_{z}—[R—CHO]

said embodiment described further herein below.

The third aspect of the overall invention relates to conjugates comprising more than two substrate units (y>2).

[Poly] units are any non-peptide polymer backbone. One aspect of the present invention relates to [Poly] units which comprise a poly(ethylene glycol) unit, herein referred to as PEG units. The PEG units of the present invention have the formula:

R¹O(CH₂CH₂O)ₙ—

wherein R¹ is hydrogen, C₁-C₄ alkyl, and mixtures thereof. The index n is from about 3 to about 200 and represents the average number of ethyleneoxy units present. In one embodiment of this aspect R¹ is hydrogen, while in another embodiment each R¹ is methyl.

In the first embodiment, the PEG units derive from poly(ethylene glycols) having the formula:

HO(CH₂CH₂O)ₙH

non-limiting examples of which include PEG 200 (n=4), PEG 300 (n=6), PEG 400 (n=9), PEG 600 (n=13), PEG 1000 (n=23), PEG 2000 (n=45), and PEG 3400 (n=77).

In another embodiment, R¹ is methyl and the poly(ethylene glycols) are therefore, methyl capped ethers, MPEG's, represented by the formula:

CH₃O(CH₂CH₂O)ₙH

non-limiting examples of which include MPEG 350 (n=7), MPEG 550 (n=12), MPEG 750 (n=16), MPEG 2000 (n=45), and MPEG 5000 (n=113).

Another aspect of the present invention relates to units having the formula:

R¹O(R²O)ₙ—

wherein R² comprises C₂-C₄ alkylene, and mixtures thereof. The index n is from about 3 to about 200 and represents the average number of alkyleneoxy units present. In one embodiment of this aspect R¹ is hydrogen, iterations of which include R² units which are a mixture of 1,2-propylene and 1,3-propylene and in another iteration R² units which are a mixture of ethylene and 1,2-propylene units. These iterations which describe mixtures of R² units can be represented by various means, for example, the formula:

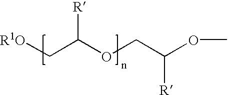

wherein R′ is hydrogen when standing for ethylene units and R′ is methyl when standing for 1,2-propylene units or, for example, by the formula:

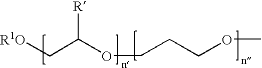

when ethylene, 1,2-propylene, and 1,3-propylene units are present; n′+n″=n. This last iteration can be considered to include block copolymers having the formula:

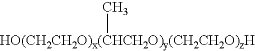

wherein x+y+z=n. A non-limiting example of this iteration includes a block copolymer having an average molecular weight of about 8400 available ex BASF as Pluronic® F-68.

This last iteration can also be considered to include block copolymers having the formula:

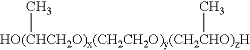

wherein x+y+z=n. A non-limiting example of this iteration includes a block copolymer having an average molecular weight of about 4550 available ex BASF as Pluronic® 10-R8.

A third aspect of the present invention as it relates to [Poly] units encompasses R² units which entirely comprise, 1,2-propylene, 1,3-propylene, 1,2-butylene, or 1,4-butylene units and wherein R¹ is hydrogen. Non-limiting examples of the iteration of this aspect wherein R¹ is hydrogen and each R²unit comprises 1,2-propylene includes [Poly] units derived from the following poly(propylene glycols); PPG 425 (n=7), PPG 725 (n=12) and PPG 1000 (n=17) all of which are available ex Aldrich Chemical.

While in another embodiment of this third aspect, each R¹ is methyl, iterations of which include R² units which are a mixture of 1,2-propylene and 1,3-propylene and in another iteration R² units which are a mixture of ethylene and 1,2-propylene units.

A fourth aspect of the [Poly] units relates to branched polymer backbones and are useful for preparing conjugates of the third overall aspect of the present invention which are conjugates having y>2 as described herein above. Said conjugates comprise units having the formula:

—O(R²O)_{n′}(R³O)_{n″}—

wherein R³ units comprise a branched unit having the formula:

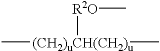

wherein R² C₂-C₄ alkylene, and each u is independently from 0 to 10 provided at least one u is not equal to 0. The branched backbone conjugates of the present invention can be conveniently derived from [Poly] backbone starting materials having the formula:

HO(R²O)_{n′}(R³O)_{n″}H

wherein the indices n′+n″=n as defined herein above.

A non-limiting example of a conjugate utilizing this aspect of the [Poly] units has the general formula:

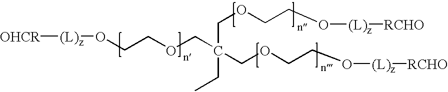

wherein n′+n″+n′″=n.

L is a linking unit which is capable of providing a link between the [Poly] backbone and the aldehyde comprising unit [R—CHO]. Linking units can be any suitable combination of atoms except highly reactive or unstable combinations, non-limiting examples of which include, O—O bonds, N—O bonds, and the like. The index z is 1 when linking units are present.

Non-limiting examples of suitable linking units includes units selected from the group consisting of:

i) —[C(R⁷)₂]p—; wherein p is from 1 to 22;

ii) —[C(R⁷)₂]ₚ(CH═CH)_{q}—; wherein p is from 0 to 12; q is from 1 to 6;

iii) —C(X)—;

iv) —OC(X)—;

v) —C(X)O—;

vi) —[C(R⁷)₂]_{q}C(X)X(R⁸O)ₚ—; wherein p is from 0 to 12; q is from 1 to 6;

vii) —(OR⁸)ₚXC(X)[C(R⁷)₂]_{q}—; wherein p is from 0 to 12; q is from 1 to 6;

viii) —C(X)NR⁷—;

ix) —C(X)R⁸C(X)—;

x) —C(X)NR⁷C(X)—;

xi) —C(X)NR⁷R⁸NR⁷C(X)—;

xii) —NR⁷C(X)—;

xiii) —NR⁷C(X)NR⁷—;

xiv) —R⁸NR⁷C(X)NR⁷—;

xv) —R⁸NR⁷C(X)NR⁷R⁸—;

xvi) —R⁸NR⁷—;

xvii) —R⁸O—;

xviii) —(R⁸)ᵤC(X)(R⁸)ᵤ—;

xix) —(R⁸)ᵤOC(O)(R⁸)ᵤ—;

xx) —(R⁸)ᵤC(O)O(R⁸)ᵤ—;

xxi) —(R⁸)ᵤOC(O)O(R⁸)ᵤ—;

wherein R⁷ is hydrogen, C₁-C₂₂ linear or branched alkyl; C₁-C₂₂ cycloalkyl; C₁-C₂₂ linear or branched fluoroalkyl; C₂-C₂₂ linear or branched alkenyl; C₆-C₂₂ aryl; C₇-C₂₂ alkylenearyl; and mixtures thereof; R⁸ is C₂-C₂₀ linear or branched, substituted or unsubstituted alkylene; C₇-C₂₀ alkylenearylene; C₆-C₂₀ substituted or unsubstituted arylene; X is oxygen, sulfur, ═NR⁷, and mixtures thereof; u is 0 or 1.

One aspect of the linking units of the present invention relates to L units which comprise a carbonyl unit. A non-limiting example of a linking group comprising conjugate of the first aspect of the invention wherein y=1 has the formula:

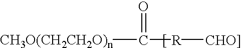

wherein n is the same as defined herein above.

Another aspect of liking units relates to urea units, for example, formed by reacting an —RCHO unit with an activated MPEG as follows:

CH₃O(CH₂CH₂O)ₙCH₂CH₂—NCO+H₂N—RCHO→

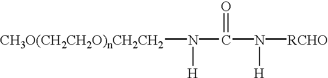

which utilizes linking unit (xiv) described herein above.

Another aspect of liking units relates to secondary amine units, for example, formed by reacting an —RCHO unit with an activated MPEG as follows:

CH₃O(CH₂CH₂O)ₙCH₂CH₂—OMs+H₂N—RCHO→CH₃O(CH₂CH₂O)ₙCH₂CH₂—NH—RCHO

which utilizes linking unit (i) described herein above.

The aldehyde comprising units of the present invention are units having the capacity to reversibly inhibit enzyme activity, said units having the formula:

—R—CHO

wherein R is a unit which facilitates the interaction of the aldehyde functionality with an enzyme. The aldehyde comprising unit is also referred to herein as “the substrate portion of the conjugate” which is capable of differential binding with enzymes. For example, one specific —RCHO unit may bind tightly with one particular protease enzyme while weakly interacting with another. Or alternatively, the substrate may bind well to all protease enzymes, as well as weakly to other enzymes, inter alia, lipase enzyme.

One aspect of R units, used herein to exemplify the relationship of R unit components, has the formula:

J—W—K

wherein the unit J is an amino acid residue having the formula:

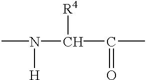

wherein for this aspect R⁴ is selected from the group consisting of 1-methylethyl, 1-methyl-propyl, 2-methylpropyl, benzyl, substituted benzyl, phenyl, substituted phenyl, and mixtures thereof. W is a spacer unit as defined herein below. K is a unit which is part of the terminal aldehyde unit and when taken together with said aldehyde unit has the formula:

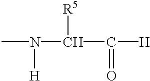

wherein for this aspect R⁵ units are selected from the group consisting of 1-methylethyl, 1-methyl-propyl, 2-methylpropyl, and mixtures thereof. It is, however, convenient to consider the R units to be taken together with the aldehyde functionality to form an —RCHO unit which has the formula:

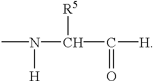

The elements J, W, and K which comprise the —RCHO units themselves function to position the substrate portion of the conjugate within the active site of the target enzyme and thereby provide a means for abating enzyme activity and a means for stabilizing enzyme comprising compositions. The formula herein above represents the broadest aspect of —RCHO substrate units which can be considered to comprise an amino acid linked to an amino aldehyde by a suitable W spacer unit. R⁴ and R⁵ are each independently a hydrophobic unit selected from the group consisting of substituted or unsubstituted:

i) phenyl;

ii) benzyl;

iii) naphthyl;

iv) linear or branched C₁-C₇ alkyl;

v) and mixtures thereof.

The carbon atoms to which R⁴ and R⁵ are bonded may be chiral or a mixture of S and R amino acids can be used to form a racemic mixture. One aspect of the aldehyde substrate units relates to compounds derived from S-amino acids and S-amino aldehydes having the formula:

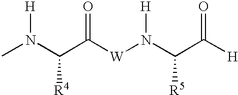

Without wishing to be limited by theory, the enzymes, which are inhibited by the conjugates of the present invention, interact primarily with the terminal aldehyde moiety. However, it has also been discovered that the relative position and size of the R⁴ and R⁵ units are important to enzyme binding activity. W units are spacer units which modulate the distance between the R⁴, R⁵ and —CHO units thereby affecting the manner in which the substrate portion of the conjugate aligns into an enzyme binding site. In general, the formulator may select any W unit which results in a substrate that is capable of providing reversible protease enzyme inhibition.

The first aspect of the present invention as it relates to the selection of W units encompasses units which are comprised of two amino acids. This selection results in a substrate portion of the conjugate which is a peptidic unit having the formula:

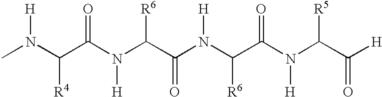

wherein each R⁶ is independently selected and can be any moiety which comprises amino acids, non-limiting examples of which include alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, valine, p-benzoyl-phenylalanine, β-(1-naphthyl)-alanine, β-(2-naphthyl)-alanine, β-cyclohexylalanine, 3,4-dichlorophenylalanine, 4-fluorophenyl-alanine, 4-nitrophenylalanine, 2-thienylalanine, 1,2,3,4-tetrahydro-isoquinoline-3-carboxylic acid, 3-benzothienylalanine, 4-cyanophenylalanine, 4-iodophenylalanine, 4-bromophenylalanine, 4,4′-biphenylalanine, ornithine, sarcosine, pentafluorophenylalanine, and β,β-diphenylalanine, and mixtures thereof.

Common amino acids result in R units selected from the group consisting of hydrogen, methyl, hydroxymethyl, 1-hydroxylethyl, 1-methylethyl, 1-methyl-propyl, 2-methylpropyl, benzyl, 4-hydroxyphenyl, 2-pyrrolidinyl, thiomethyl, (methylthio)methyl, carboxymethyl, carboxyethyl, 3-indolylmethyl, 4-aminobutyl, 3-guanidinopropyl, 1H-3-imidazolyl-, and mixtures thereof.

One embodiment of this aspect, which selects W units comprising two amino acids, has the formula:

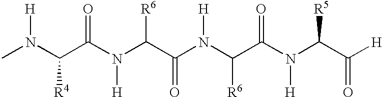

wherein each R⁶ is hydrogen, methyl, ethyl, and mixtures thereof. One iteration of this embodiment which utilizes three chiral amino acids and a chiral amino aldehyde results in a series of polymer conjugates having the formula:

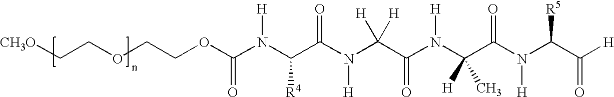

wherein W derives from the amino acids glycine and alanine (Gly-Ala) and L is a carbonyl linking unit.

The following are non-limiting examples of the reversible enzyme inhibitors of the present invention.

TABLE I

| n¹ | R⁴ | R⁵ | n¹ | R⁴ | R⁵ |
|---|---|---|---|---|---|
| 44 | benzyl | 1-Me-propyl | 112 | benzyl | 1-Me-propyl |
| 44 | benzyl | 2-Me-propyl | 112 | benzyl | 2-Me-propyl |
| 44 | benzyl | 1-Me-ethyl | 112 | benzyl | 1-Me-ethyl |
| 44 | benzyl | methyl | 112 | benzyl | methyl |
| 44 | benzyl | ethyl | 112 | benzyl | ethyl |
| 44 | 4-HO-phenyl | 1-Me-propyl | 112 | 4-HO-phenyl | 1-Me-propyl |
| 44 | 4-HO-phenyl | 2-Me-propyl | 112 | 4-HO-phenyl | 2-Me-propyl |
| 44 | 4-HO-phenyl | 1-Me-ethyl | 112 | 4-HO-phenyl | 1-Me-ethyl |
| 44 | 4-HO-phenyl | methyl | 112 | 4-HO-phenyl | methyl |
| 44 | 4-HO-phenyl | ethyl | 112 | 4-HO-phenyl | ethyl |

¹The index n represents the average number of units present.
Table I relates to conjugates wherein R⁴ comprises an aryl or substituted aryl unit, for example, an amino acid derived from phenylalanine or tyrosine.
However, the R⁵ unit may also comprise a substituted or unsubstituted aryl unit. The following non-limiting examples relate to a second iteration of the embodiment of W units derived from (Gly-Ala).

TABLE II

| n¹ | R⁴ | R⁵ | n¹ | R⁴ | R⁵ |
|---|---|---|---|---|---|
| 44 | benzyl | phenyl | 112 | benzyl | phenyl |
| 44 | benzyl | 4-HO-phenyl | 112 | benzyl | 4-HO-phenyl |
| 44 | benzyl | 4-Me-phenyl | 112 | benzyl | 4-Me-phenyl |
| 44 | benzyl | benzyl | 112 | benzyl | benzyl |
| 44 | benzyl | 4-HO-benzyl | 112 | benzyl | 4-HO-benzyl |
| 44 | benzyl | 4-Me-benzyl | 112 | benzyl | 4-Me-benzyl |
| 44 | 4-HO-phenyl | phenyl | 112 | 4-HO-phenyl | phenyl |
| 44 | 4-HO-phenyl | 4-HO-phenyl | 112 | 4-HO-phenyl | 4-HO-phenyl |
| 44 | 4-HO-phenyl | 4-Me-phenyl | 112 | 4-HO-phenyl | 4-Me-phenyl |
| 44 | 4-HO-phenyl | benzyl | 112 | 4-HO-phenyl | benzyl |
| 44 | 4-HO-phenyl | 4-HO-benzyl | 112 | 4-HO-phenyl | 4-HO-benzyl |
| 44 | 4-HO-phenyl | 4-Me-benzyl | 112 | 4-HO-phenyl | 4-Me-benzyl |

¹The index n represents the average number of units present.
Table II relates to conjugates wherein R⁴ comprises an aryl or substituted aryl unit, for example, an amino acid derived from phenylalanine or tyrosine and R⁵ comprises an amino acid having an aryl unit.
However, the R⁵ unit may also comprise a substituted or unsubstituted alkyl unit. The following non-limiting examples relate to a third iteration of the embodiment of W units derived from (Gly-Ala).

TABLE III

| n¹ | R⁴ | R⁵ | n¹ | R⁴ | R⁵ |
|---|---|---|---|---|---|
| 44 | 1-Me-propyl | benzyl | 112 | 1-Me-propyl | benzyl |
| 44 | 2-Me-propyl | benzyl | 112 | 2-Me-propyl | benzyl |
| 44 | 1-Me-ethyl | benzyl | 112 | 1-Me-ethyl | benzyl |
| 44 | methyl | benzyl | 112 | methyl | benzyl |
| 44 | ethyl | benzyl | 112 | ethyl | benzyl |
| 44 | 1-Me-propyl | 4-HO-phenyl | 112 | 1-Me-propyl | 4-HO-phenyl |
| 44 | 2-Me-propyl | 4-HO-phenyl | 112 | 2-Me-propyl | 4-HO-phenyl |
| 44 | 1-Me-ethyl | 4-HO-phenyl | 112 | 1-Me-ethyl | 4-HO-phenyl |
| 44 | methyl | 4-HO-phenyl | 112 | methyl | 4-HO-phenyl |
| 44 | ethyl | 4-HO-phenyl | 112 | ethyl | 4-HO-phenyl |

¹The index n represents the average number of units present.
Table III relates to conjugates wherein R⁴ comprises an alkyl or substituted alkyl unit, for example, an amino acid derived from leucine or isoleucine and R⁵ comprises an amino acid having an aryl unit.
However, both the R⁴ unit and R⁵ unit may comprise a substituted or unsubstituted alkyl unit. The following non-limiting examples relate to a fourth iteration of the embodiment of W units derived from (Gly-Ala).

TABLE IV

| n¹ | R⁴ | R⁵ | n¹ | R⁴ | R⁵ |
|---|---|---|---|---|---|
| 44 | 1-Me-propyl | 1-Me-propyl | 112 | 1-Me-propyl | 1-Me-propyl |
| 44 | 2-Me-propyl | 2-Me-propyl | 112 | 2-Me-propyl | 2-Me-propyl |
| 44 | 1-Me-ethyl | 1-Me-ethyl | 112 | 1-Me-ethyl | 1-Me-ethyl |
| 44 | methyl | methyl | 112 | methyl | methyl |
| 44 | ethyl | ethyl | 112 | ethyl | ethyl |
| 44 | 1-Me-propyl | 1-Me-propyl | 112 | 1 -Me-propyl | 1-Me-propyl |
| 44 | 2-Me-propyl | 2-Me-propyl | 112 | 2-Me-propyl | 2-Me-propyl |
| 44 | 1-Me-ethyl | 1-Me-ethyl | 112 | 1-Me-ethyl | 1-Me-ethyl |
| 44 | methyl | methyl | 112 | methyl | methyl |
| 44 | ethyl | ethyl | 112 | ethyl | ethyl |

¹The index n represents the average number of units present.
A second aspect of the W unit according to the present invention relates to the selection of units which are isosteric or which provide sufficient special orientation between the R⁴ unit and R⁵ unit within an enzyme active site.
One embodiment of the aspect of the W unit relates to 4-aminobenzoic acid, 3-aminomethylbenzoic acid, and 2-aminoethylbenzoic acid, each of which can be further substituted.

Iterations of this aspect of W units include units selected from the group consisting of:

i) 4-aminobenzoic acids having the formula:

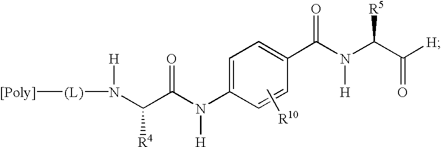

ii) 3-aminomethylbenzoic acids having the formula:

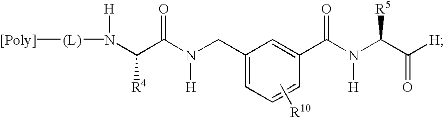

iii) 2-aminoethylbenzoic acids having the formula:

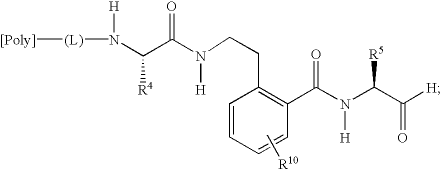

wherein R¹⁰ can be any moiety which can substitute for hydrogen as defined herein above.

However another embodiment of this aspect relates to W units wherein the carboxy terminus is extended from the aryl ring, for example a conjugate having the general formula:

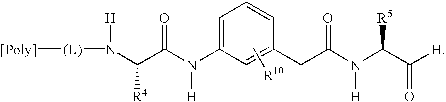

Another aspect of the W units relates to non-aryl carbocyclic and heterocyclic units suitable as a unit which provides the proper alignment of R⁴ and R⁵ within an enzyme receptor site. Non-limiting examples of rings, which can substitute for phenyl when properly substituted in a like manner of the second aspect of the W units, include cyclopentane, cyclohexane, furan, thiophene, pyrrole, imidazole, pyran, thiazole, pyridine, pyridazine, pyrimidine, pyrazine, piperidine, piperazine, morpholine, and the like.

As described herein above, the second overall aspect of the present invention relates to conjugates having two substrate units (y=2). Conjugates of this type can be readily formed from [Poly] starting materials having two reactive sites, for example PEG units described herein above. The first embodiment of [Poly] units relates to PEG units derive from poly(ethylene glycols) having the formula:

HO(CH₂CH₂O)ₙH

which can be used to form second aspect conjugates having the formula:

OHCR—(L)_{z}—O(CH₂CH₂O)ₙ—(L)_{z}—RCHO

wherein the units R and L and the indices n and z are the same as defined hereinabove.

A non-limiting example of one embodiment of this aspect has the general formula:

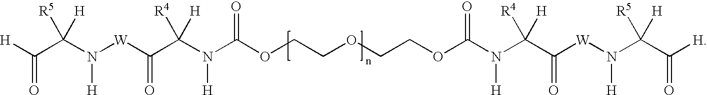

The following are non-limiting examples of procedures for preparing the conjugates of the present invention.

Conjugates of the present invention can be prepared by a variety of routes, starting with an activated PEG polymer and a suitable peptide aldehyde precursor. The peptide aldehyde precursor may be a peptide alcohol, such that the conjugate is converted to the desired aldehyde by oxidation. Alternatively, the peptide aldehyde precursor may be in a higher oxidation state, such as a Weinreb amide, which is reduced to the aldehyde oxidation state. Still another method is to use a peptide aldehyde protected as the acetal. Acid hydrolysis of the resulting PEG-peptide acetal conjugate affords the desired aldehyde. The intermediate and final polymer conjugates are conveniently isolated by pouring a solution of the conjugate into a suitable non-solvent. Non-limiting examples of non-solvents include diethyl ether, diisopropyl ether, petroleum ether, hexane, and pentane. The following are non-limiting examples of procedures for preparing the conjugates of the present invention.

### EXAMPLE 1

Preparation of N-Boc-Phe-Gly-Ala-Leu-OCH₃

N-Boc-Phe-Gly (6.38 g, 19.8 mmol), HCl Ala-Leu-OCH₃ (5 g, 19.8 mmol), HOBT (3.24 g, 21.8 mmol), and triethylamine (3.0 mL, 21.8 mmol) are dissolved in dichloromethane (1 33 mL). A solution of dicyclohexylcarbodiimide (DCC) (4.49 g, 21.8 mmol) in dichloromethane is added to the reaction mixture and the reaction mixture is stirred for at least 2 h. The solution is filtered to remove the solid dicyclohexylurea which forms and the dichloromethane solution is washed with water, NaHCO₃, and 10% citric acid. The organic layer is dried over magnesium sulfate, the solution is filtered and the solvent is removed in vacuo to give the desired product, N-Boc-Phe-Gly-Ala-Leu-OCH₃.

Preparation of Phe-Gly-Ala-Leucinol Hydrochloride

Boc-Phe-Gly-Ala-Leu-OCH₃ (1.50 g, 2.88 mmol) is dissolved in 50 mL of 1:1 THF/ EtOH and stirred in a 100 mL RB flask under argon until the solution clears. Freshly-ground, anhydrous calcium chloride (0.64 g, 5.77 mmol) is added to the reaction and stirred until most of the solid dissolves. The flask is cooled to approximately −15° C. using a bath of ice/ethanol/dry ice. Sodium borohydride (0.54 g, 11.54 mmol) is added in increments over a 15 minute period maintaining the −15° C. temperature. The reaction is stirred for 1-2 hours until the starting material is no longer detected by TLC analysis (5% methanol in methylene chloride, silica gel). The reaction is stirred with 100 mL of 1.0 N HCl for 1 hour to quench. THF and ethanol are removed on a rotovap and the aqueous mixture is extracted with ethyl acetate (4×50 mL). The combined ethyl acetate solution is washed sequentially with sodium bicarbonate followed by brine and then dried over magnesium sulfate and is filtered. The solvent is evaporated in vacuo to provide Boc-Phe-Gly-Ala-Leucinol. The solid is dissolved in a minimum amount of dicloromethane and 2.0 M HCl in diethyl ether is added. The solution is stirred for a minimum of 6 hours, then the precipitate is filtered and is washed with diethyl ether. Residual solvent is removed in vacuo and Phe-Gly-Ala-Leucinol hydrochloride salt is isolated as hygroscopic, off-white solid.

Preparation of MPEG-Phe-Gly-Ala-Leucinol

Phe-Gly-Ala-Leucinol (hydrochloride salt; 5.6 g, 13.0 mmol) is dissolved in dry pyridine (20 eq), diluted with dichloromethane to 10% solids, and stored overnight on 4 Å molecular sieves.

While the peptide is drying, the methoxy-PEG is activated by reaction with phosgene. M-PEG-OH (50.0 g, 10.0 mmol) is dissolved in toluene at 40° C. Under an inert atmosphere, a solution of phosgene in toluene (1.6 mL, 1.93M) is added. After the addition, the reaction mixture is stirred for 12-18 h under an inert atmosphere at 40° C. The reaction is sparged sufficiently to drive off excess phosgene and the resulting M-PEG chloroformate solution is added to the solution of tetrapeptide alcohol. The mixture is stirred for 4 to 6 hours, diluted with dichloromethane and filtered to remove molecular sieves and other insoluble material. The filtrate is washed with water to remove pyridine, pyridine hydrochloride, and other water-soluble impurities. The organic layer is concentrated in vacuo to 200 mL, dried over MgSO₄, and then poured onto 2L dry diisopropyl ether. The resulting precipitate of MPEG-Phe-Gly-Ala-Leucinol is collected by filtration and dried under vacuum.

MPEG-Phe-Gly-Ala-Leu-H

MPEG-Phe-Gly-Ala-Leucinol (43 g) is dissolved in 93 mL dichloromethane. A solution of sodium bromide in water (0.9M, 900 mg) is added and the solution is cooled in an ice bath. Cold TEMPO (6.96 meq, 8.7 mg) is added followed by KHCO₃ (5.8 eq, 6.4 g). Bleach (6% NaOCl, 1.1 eq, 10.9 g) is cooled in an ice bath and added to the solution. The reaction is quenched by addition of an aqueous solution of Na₂S₂O₃ (1.46 eq, 1.8 g, 0.5M). The solution is poured into a separatory funnel and washed with water. The resulting mixture is poured through glass wool into another separatory funnel and the layers are easily separated. The organic layer is collected and concentrated to 200 mL on a rotovap, dried over MgSO₄ and poured onto 2L diisopropyl ether. The resulting precipitate is isolated by vacuum filtration.

Formulations

The use of enzymes, especially in personal care compositions is known. The enzymes, particularly protease enzymes, are believed to provide a desquamatory benefit wherein the outer layers of skin are removed thereby providing a fresh health skin surface. The compositions of the present invention comprise one or more enzymes and one or more enzyme inhibitors according to the present invention depending upon the type of formulation or, importantly, the temperature of exposure (e.g. storage conditions).

The use of an enzyme inhibitor not only acts to stabilize the enzyme until it is needed, but the inhibitor stabilizes the enzyme towards auto-digestion of itself and other components. As a guideline for preparing compositions comprising protease enzymes and enzyme inhibitors, the relative inhibition constant, Kᵢ, of the inhibitor is taken into consideration when formulating said compositions. Inhibition constants need not be exact such that approximate values are readily adaptable to formulation parameters. One convenient method for measuring and obtaining Kᵢ values is described in “Kinetics of Subtilisin and Thiosubtilisin”, M. Philipp et al., Molecular & Cellular Biochemistry, 51, pp. 5-32 (1983).

One embodiment of the present invention utilizes inhibitors with a Kᵢ of from 10 nM to 25,000 nM, while another aspect includes a narrower range of from 50 nM too 5,000 nM. A typical range which is convenient for most applications comprises a Kᵢ within the range of from 100 nM to 1,000 nM.

Depending upon the Kᵢ and respective molecular weights of the enzyme and inhibitor, the amount of inhibitor present will vary, however, it is convenient for the formulator to begin within the range of from 2 moles of inhibitor to 1 mole of enzyme to a ratio of 10 mole of inhibitor to 1 moles of enzyme.

For the purposes of the present invention the inhibition constant, Kᵢ, is defined herein as:

$${K}_{i} = \frac{[ E ] [ I ]}{[ EI ]}$$

wherein [E], [I ], and [EI ] are the concentrations of the unbound enzyme, the free inhibitor, and the enzyme-inhibitor complex respectively.

The compositions of the present invention comprise:

a) from about 0.001% to about 0.5% by weight, one or more protease enzymes;

b) from about 0.001% to about 0.5% by weight, one or more protease enzyme inhibitors according to the present invention; and

c) the balance carriers and adjunct ingredients.

Non-limiting examples of adjunct ingredients include polyhydric alcohols, inter alia, glycerine; osmo-protectants; lyotropic stabilizers; pH modifiers and buffering agents; polymeric thickeners; skin active agents, inter alia, vitamins; emollients, silicone oils, emulsifiers, surfactants, anti-microbial; sun-screening agents; and the like

The following are non-limiting examples of compositions according to the present invention.

TABLE V
MOISTURIZING BODY WASH
weight %

| Ingredients | 2 | 3 | 4 | 5 |
|---|---|---|---|---|
| Glycerin | 4.0 | — | — | — |
| PEG-6 Caprylic/Capric Glycerides | 4.0 | — | — | — |
| Palm Kernal Fatty acids | 3.0 | — | — | — |
| Sodium Laureth-3 Sulphate | 45.0 | 12.0 | 15.0 | 8.0 |
| Cocamide MEA | 3.0 | — | — | — |
| Cocamidopropyl betaine | — | 8.0 | 10.0 | 15.0 |
| Sodium Lauroamphoacetate | 25.0 | | | |
| APG Glucoside¹ | — | — | 2.0 | 1.0 |
| Soybean Oil | 10.0 | — | — | — |
| Polyquaternium-10 (JR30M) | 0.7 | 0.25 | — | — |
| Polyquaternium-7 (Mackam 55) | — | — | — | 0.7 |
| Protease | 0.1 | 0.025 | 0.05 | 0.1 |
| Conjugate² | 0.1 | 0.025 | 0.05 | 0.1 |
| Deionized water | Balance | Balance | Balance | Balance |
| pH | 7 | 6.5 | 7 | 8.5 |

¹Plantacare 2000 ex Cognis.
²Polymer conjugate according to Example 1.

TABLE VI
BODY LOTION
weight %

| Ingredients | 6 | 7 | 8 | 9 |
|---|---|---|---|---|
| Glycerine | 8.0 | 8.0 | 10.0 | 12.0 |
| Isohexadecane | 3.0 | 3.0 | 3.0 | 6.0 |
| Niacinamide | — | 3.0 | 5.0 | 6.0 |
| Isopropyl isostearate | 3.0 | 3.0 | 3.0 | 3.0 |
| Sepigel 305¹ | 3.0 | 3.0 | 3.0 | 3.0 |
| Petrolatum | 4.0 | 4.0 | 4.0 | 2.0 |
| Nylon 12 | 2.0 | 2.0 | 2.5 | 2.5 |
| Dimethicone² | 2.0 | 2.0 | 2.5 | 2.5 |
| Sucrose polycottonseed oil | 1.5 | 1.5 | 1.5 | 1.5 |
| Stearyl Alcohol 97% | 1.0 | 1.0 | 1.0 | 1.0 |
| D-panthenol | 1.0 | 1.0 | 1.0 | 1.0 |
| D,L-□-tocopherol acetate | 1.0 | 1.0 | 1.0 | 1.0 |
| Cetyl Alcohol 95% | 0.5 | 0.5 | 0.5 | 1.0 |
| Behynyl alcohol | 1.0 | 1.0 | 1.0 | 0.5 |
| EMULGADE PL 68/50 | 0.4 | 0.4 | 0.5 | 0.5 |
| Stearic acid | 0.15 | 0.15 | 0.15 | 0.15 |
| PEG-100 stearate³ | 0.15 | 0.15 | 0.15 | 0.15 |
| Protease | 0.005 | 0.005 | 0.025 | 0.1 |
| Conjugate⁴ | 0.005 | 0.005 | 0.025 | 0.1 |
| Deionized water | Balance | Balance | Balance | Balance |
| pH | 7 | 7 | 7.5 | 7 |

¹Comprises .pPolyacrylamide, Isoparaffin, and Laureth-7 ex Seppic.
²DC1403 ex Dow Corning.
³MYRJ 59 ex Uniqema.
⁴Polymer conjugate according to Example 1.

TABLE VII
MOISTURIZING FACIAL CREAM OR LOTION
weight %

| Ingredients | 10 | 11 |
|---|---|---|
| Glycerine | 12.0 | 5.0 |
| Isohexadecane | 5.0 | 5.0 |
| Niacinamide | 5.0 | 7.0 |
| Isopropyl isostearate | 2.0 | 2.0 |
| Sepigel 305¹ | 3.0 | 3.0 |
| PEG 4000 | — | 10.0 |
| Polymethylsilsesquioxane | 2.0 | 2.0 |
| Dimethicone² | 3.0 | 2.0 |
| Sucrose polycottonseed oil | 1.0 | 1.0 |
| Stearyl Alcohol 95% | 0.5 | 0.5 |
| D-panthenol | 1.0 | 1.0 |
| D,L-□-tocopherol acetate | 1.0 | 1.0 |
| Cetyl Alcohol 95% | 1.0 | 1.0 |
| Cetearyl glucoside | 0.5 | 0.5 |
| Titanium dioxide | 0.3 | 0.3 |
| Stearic acid | 0.15 | 0.15 |
| PEG-100 stearate³ | 0.15 | 0.15 |
| Protease | 0.05 | 0.05 |
| Conjugate∝ | 0.05 | 0.05 |
| Deionized water | balance | balance |
| pH | 7 | 7 |

¹Comprises .pPolyacrylamide, Isoparaffin, and Laureth-7 ex Seppic.
²DC1403 ex Dow Corning.
³MYRJ 59 ex Uniqema.
⁴Polymer conjugate according to Example 1.

TABLE VIII
FACIAL MOISTURIZING CREAM
weight %

| Ingredients | 12 | 13 | 14 |
|---|---|---|---|
| Glycerine | 3.0 | 5.0 | 10.0 |
| Petrolatum | 3.0 | 3.0 | — |
| Cetyl Alcohol 95% | 1.5 | 1.5 | 1.0 |
| Dimethicone copolyol¹ | 2.0 | 2.0 | 2.0 |
| Isopropyl palmitate | 1.0 | 1.0 | 0.5 |
| Carbomer 954 | 0.7 | 0.7 | 0.7 |
| Dimethicone² | 1.0 | 1.0 | 1.0 |
| Stearyl Alcohol 97% | 0.5 | 0.5 | 1.0 |
| Stearic acid | 0.1 | 0.1 | 0.1 |
| PEG-100 stearate³ | 0.1 | 0.1 | 0.1 |
| Titanium dioxide | 0.3 | 0.3 | 0.3 |
| Protease | 0.005 | 0.025 | 0.1 |
| Conjugate⁴ | 0.005 | 0.025 | 0.1 |
| Deionized water | Balance | Balance | Balance |
| pH | 7 | 7 | 7.5 |

¹DC 3225C ex Dow Corning.
²DC 200/350 cs ex Dow Corning.
³MYRJ 59 ex Uniqema.
⁴Polymer conjugate according to Example 1.

What is claimed is:

1. An enzyme inhibitor conjugate having the formula:
[Poly][(L)_{z}—[R—CHO]┐_{y}
[Poly] is a water-soluble, non-peptidic polymer component, L is an optionally present linking group, and RCHO is a substrate capable of interacting with one or more enzymes to reversibly inhibit said enzyme; y has the value of at least 1; z has the value 0 or 1.

2. A conjugate according to claim 1 having the formula:
[Poly]—(L)_{z}—[R—CHO].

3. A conjugate according to claim 2 wherein [Poly] has the formula:
R¹O(CH₂CH₂O)ₙ—
wherein R¹ is hydrogen, C₁-C₄ alkyl, and mixtures thereof; the index n is from about 3 to about 200.

4. A conjugate according to claim 3 wherein R¹ is methyl.

5. A conjugate according to claim 4 wherein n is from 7 to 20.

6. A conjugate according to claim 4 wherein n is from 20 to 100.

7. A conjugate according to claim 2 wherein [Poly] has the formula:
R¹O(R²O)ₙ—
wherein R¹ is hydrogen, C₁-C₄ alkyl, and mixtures thereof; R² comprises C₂-C₄ alkylene, and mixtures thereof. The index n is from about 3 to about 200.

8. A conjugate according to claim 7 wherein R¹ is methyl.

9. A conjugate according to claim 7 wherein R² units are a mixture of 1,2-propylene and 1,3-propylene.

10. A conjugate according to claim 2 comprising a linking group selected from the group consisting of:
i) —[C(R⁷)₂]ₚ—; wherein p is from 1 to 22;
ii) —[C(R⁷)₂]ₚ(CH═CH)_{q}—; wherein p is from 0 to 12; q is from 1 to 6;
iii) —C(X)—;
iv) —OC(X)—;
v) —C(X)O—;
vi) —[C(R⁷)₂]_{q}C(X)X(R⁸O)ₚ—; wherein p is from 0 to 12; q is from 1 to 6;
vii) —(OR⁸)ₚXC(X)[C(R⁷)₂]_{q}—; wherein p is from 0 to 12; q is from 1 to 6;
viii) —C(X)NR⁷—;
ix) —C(X)R⁸C(X)—;
x) —C(X)NR⁷C(X)—;
xi) —C(X)NR⁷R⁸NR⁷C(X)—;
xii) —NR⁷C(X)—;
xiii) —NR⁷C(X)NR⁷—;
xiv) —R⁸NR⁷C(X)NR⁷—;
xv) —R⁸NR⁷C(X)NR⁷R⁸—;
xvi) —R⁸NR⁷—
xvii) —R⁸O—;
xviii) —(R⁸)ᵤC(X)(R⁸)ᵤ—;
xix) —(R⁸)ᵤOC(O)(R⁸)ᵤ—;
xx) —(R⁸)ᵤC(O)O(R⁸)ᵤ—;
xxi) —(R⁸)ᵤOC(O)O(R⁸)ᵤ—;
wherein R⁷ is hydrogen, C₁-C₂₂ linear or branched alkyl; C₁-C₂₂ cycloalkyl; C₁-C₂₂ linear or branched fluoroalkyl; C₂-C₂₂ linear or branched alkenyl; C₆-C₂₂ aryl; C₇-C₂₂ alkylenearyl; and mixtures thereof; R⁸ is C₂-C₂₀ linear or branched, substituted or unsubstituted alkylene; C₇-C₂₀ alkylenearylene; C₆-C₂₀ substituted or unsubstituted arylene; X is oxygen, sulfur, ═NR⁷, and mixtures thereof; u is 0 or 1.

11. A conjugate according to claim 10 wherein L is —[C(R⁷)₂]ₚ—; —NR⁷C(O)—; —C(O)—; and mixtures thereof.

12. A conjugate according to claim 11 wherein L is —C(O)—.

13. A conjugate according to claim 2 wherein —RCHO has the formula: 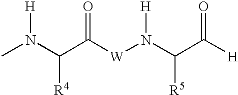
wherein R⁴ and R⁵ are each independently selected from the group consisting of substituted or unsubstituted:
i) phenyl;
ii) benzyl;
iii) naphthyl;
iv) linear or branched C₁-C₇ alkyl;
v) and mixtures thereof;
W units are spacer units which modulate the distance between the R⁴, R⁵ and —CHO units.

14. A conjugate according to claim 13 having the formula: 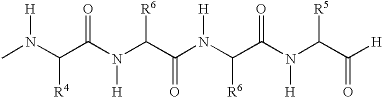
wherein each R⁶ is independently selected from the group consisting of hydrogen, methyl, hydroxymethyl, 1-hydroxylethyl, 1-methylethyl, 1-methylpropyl, 2-methylpropyl, benzyl, 4-hydroxyphenyl, 2-pyrrolidinyl, thiomethyl, (methylthio)methyl, carboxymethyl, carboxyethyl, 3-indolylmethyl, 4-aminobutyl, 3-guanidinopropyl, 1H-3-imidazolyl-, and mixtures thereof.

15. A conjugate according to claim 14 wherein R⁶ is hydrogen, methyl, 1-methylethyl, 1-methylpropyl, 2-methylpropyl, and mixtures thereof.

16. A conjugate according to claim 15 wherein R⁶ is hydrogen, methyl, and mixtures thereof.

17. A conjugate according to claim 13 wherein R⁴ is substituted or unsubstituted benzyl.

18. A conjugate according to claim 13 wherein R⁵ is hydrogen, methyl, 1-methylethyl, 1-methylpropyl, 2-methylpropyl, and mixtures thereof.

19. A conjugate according to claim 18 wherein R⁵ is 1-methylethyl, 1-methylpropyl, 2-methylpropyl, and mixtures thereof.

20. A conjugate according to claim 13 wherein —RCHO has the formula: 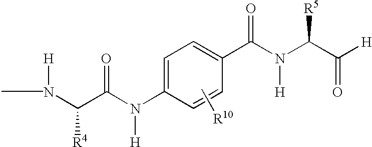
wherein R¹⁰ is a unit which can substitute for hydrogen.

21. A conjugate according to claim 13 wherein —RCHO has the formula: 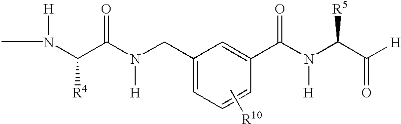
wherein R¹⁰ is a unit which can substitute for hydrogen.

22. A conjugate according to claim 13 wherein —RCHO has the formula: 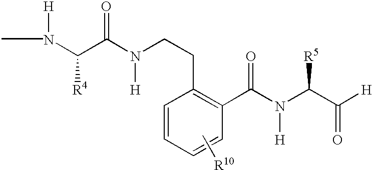
wherein R¹⁰ is a unit which can substitute for hydrogen.

23. An enzyme inhibitor conjugate having the formula: 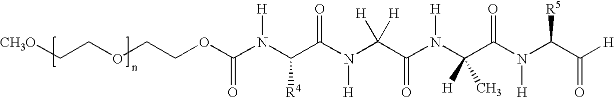
wherein R⁴ and R⁵ are each independently selected from the group consisting of substituted or unsubstituted:
i) phenyl;
ii) benzyl;
iii) naphthyl;
iv) linear or branched C₁-C₇ alkyl;
v) and mixtures thereof;
n is from 3 to 200.

24. A conjugate according to claim 23 wherein R⁴ is substituted or unsubstituted benzyl.

25. A conjugate according to claim 23 wherein R⁵ is 1-methylethyl, 1-methylpropyl, 2-methylpropyl, and mixtures thereof.

26. A conjugate according to claim 23 wherein n is 44 or 112.

27. An enzyme inhibitor conjugate having the formula:

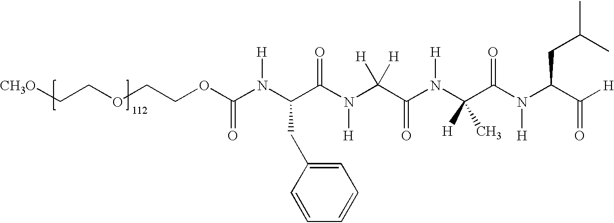

28. An enzyme inhibitor conjugate having the formula:

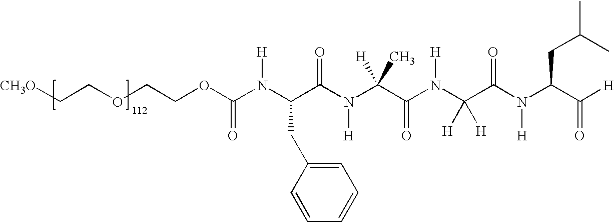

29. An enzyme inhibitor conjugate having the formula:

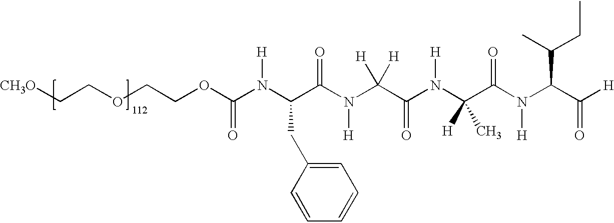

30. An enzyme inhibitor conjugate having the formula:

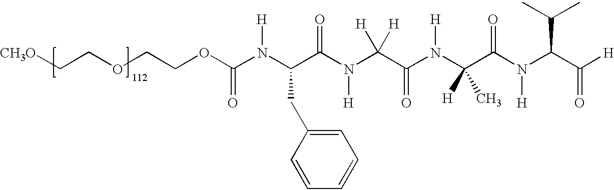

31. An enzyme inhibitor conjugate having the formula: 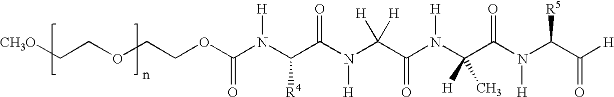
wherein R⁴ is substituted or unsubstituted benzyl, R⁵ is 1-methylethyl, 1-methylpropyl, 2-methylpropyl, or mixtures thereof; n is an integer representing the average molecular weight of an ethyleneoxy unit, said integer selected from the group consisting of 7, 122, 16, 45, and 113.

32. A composition comprising:
a) from about 0.001% to about 0.5% by weight, one or more protease enzymes;
b) from about 0.001% to about 0.5% by weight, one or more enzyme inhibitor conjugate having the formula:
[Poly][(L)_{z}—[R—CHO]┐_{y}
[Poly] is a water-soluble, non-peptidic polymer component, L is an optionally present linking group, and RCHO is a substrate capable of interacting with one or more enzymes to reversibly inhibit said enzyme; y has the value of at least 1; z has the value 0 or 1; and
c) the balance carriers and adjunct ingredients.
